(19) United States
(12) Patent Application Publication
(10) Pub. No.: US 2016/0193474 A1
(43) Pub. Date: Jul. 7, 2016

(54) AUTOMATED BATTERY INDICATION AND FEEDBACK SYSTEM BASED ON ENVIRONMENTAL CONDITIONS AND USE DATA FOR IMPROVED MANAGEMENT AND RELIABILITY

(71) Applicant: KONINKLIJKE PHILIPS N.V., Eindhoven (NL)

(72) Inventor: GEORGE PAUL GUMBRELL, WORCESTER, MA (US)

(21) Appl. No.: 14/911,877

(22) PCT Filed: Aug. 11, 2014
(86) PCT No.: PCT/IB2014/063844

§ 371 Date: Feb. 12, 2016

Related U.S. Application Data
(60) Provisional application No. 61/865,314, filed on Aug. 13, 2013.

(51) Int. Cl. A61N 1/39; H01M 10/48; G01R 31/36; H01M 10/42

(52) U.S. Cl. CPC A61N 1/3993; H01M 10/425; H01M 10/488; A61N 1/3975; G01R 31/3679; G01R 31/3682; G01R 31/3606; H01M 2010/4271; H01M 2010/4278

(57) ABSTRACT

A battery having a display indicating an amount of time the battery can be used before needing to be recharged and/or replaced. The amount of time can be displayed in minutes. An end of life indicator indicating whether the battery should be replaced can also be provided on the battery. The battery can determine the amount of time and an end of life indication based on environmental conditions and/or use. The battery can include sensors for measuring and/or monitoring environmental conditions, as well as a global positioning system (GPS) transponder. The battery can also include a communication link for transmitting data to a central location, a specified location and/or a pre-selected location, which data can include the amount of time the battery can be used before needing to be recharged and/or replaced. The battery can be coupled to and configured to power a device. Particular advantages can be realized with medical devices, especially defibrillators and other Advanced Life Support Devices which are typically exposed to a wide range of end user use models and environmental conditions.

The present invention relates to, e.g., batteries, such as may be used in/with medical devices including defibrillators and monitors, and more particularly to novel and inventive automated battery indication and feedback systems based on environmental conditions and/or use data for improved battery/device management and reliability.

Batteries are increasingly becoming an integral part of hospital healthcare, home healthcare and remote healthcare environments for medical devices. Today, just about every medical product has a primary battery for power and/or a backup battery for emergency power when the device is unplugged or experiences loss of power. With the increase of battery dependent medical devices, there are many different batteries types, runtimes, chemistries, sizes, shapes, voltages, capacities, and different preventive maintenance processes in the healthcare environment. This often leads to complexity and confusion over battery charge state and when to remove the battery from service.

Currently, batteries essentially lack the ability to provide a universally clear indication of charge state, e.g., “in minutes or hours” of product use. Today, some batteries may provide a LED “gas gauge” indicating percentage charged. For example, 5 LEDs may indicate percentage charge in approximately 20% increments. While this may seem to be helpful, it may not be clear what the LEDs, or even the percentage of time means, especially when there are many different products and batteries being used and/or managed, such as in a hospital or elsewhere. In a power outage or power interruption, this can only add to the confusion, particularly when multiple battery powered products and batteries need to be managed.

Batteries also currently lack the capability themselves to indicate an END OF LIFE condition so the user can know when the battery should be discarded/replaced based on, e.g., use and/or environmental conditions and thus reduce costs and risk of battery failure. Most medical device manufacturers define a battery life in which the user must replace the battery based on only time (e.g., typically 2 to 3 years), regardless of use and environmental conditions. This typically requires tracking, monitoring, testing and generally becomes expensive. If one replaces the battery to soon, they are increasing their long term operational costs. If one replaces the battery too late, patient safety can be an issue.

Additionally, batteries lack the ability to themselves notify, e.g., call centers or a biomed department of a hospital of low battery conditions (including, e.g., indicating a device is unplugged or not charging), battery fault, and/or battery end of life condition, much less being able to identify and communicate its location. For example, a typical hospital may have a few hundred different pieces of equipment and a few thousand different batteries distributive throughout the hospital, home and remote sites. Having the ability to accurately, timely and efficiently monitor “battery health” can enable the monitoring and maintaining of batteries, and being prepared when emergency backup power is needed.

Thus, there exists a need for more accurate, timely and efficiently monitoring of batteries, allowing for improved battery management and service (e.g., recharging and discarding/replacement), especially for users/entities having numerous batteries in various locations.

Disclosed and described herein, for example, are exemplary embodiments of the present invention, which, as one having ordinary skill in the art shall appreciate in view of the teachings herein, can be used together or separately to overcome the above-described needs and related challenges.

In accordance with exemplary embodiments of the present invention, provided is a battery having a display indicating an amount of time the battery can be used before needing to be recharged and/or replaced. The amount of time can be displayed in minutes. The display can provide an end of life indicator indicating whether the battery should be replaced. The battery can determine the amount of time and an end of life indication based on environmental conditions and/or use.

The exemplary battery can also include sensors for measuring and/or monitoring environmental conditions. It is also possible that the battery include a global positioning system (GPS) transponder. Further, the exemplary battery can include a communication link for transmitting data to a central location, a specified location and/or a pre-selected location. The data can include, e.g., the amount of time the battery can be used before needing to be recharged and/or replaced.

In accordance with another exemplary embodiment of the present invention, provided is a system including a device and a battery coupled to the device and configured to power the device. The battery includes a display indicating an amount of time the battery can be used to power the device before needing to be recharged or replaced. The display can be configured to display the amount of time in minutes.

It is also possible that the display be configured to display an end of life condition. The end of life condition can be a number of recharge cycles remaining before the battery should be replaced. Further, the display can be structured and configured to display the end of life condition as an indication that the battery should be replaced. The battery can determine the amount of time based on environmental conditions and/or use. Additionally, the battery can include sensors for measuring and/or monitoring environmental conditions. It is also possible that the battery is structured and configured to measure and/or monitor use.

The exemplary system can further include a communication link for transmitting data to a central location, a specified location and/or a pre-selected location. The communication link can be coupled directly to the battery. The communication link can also be structured and configured to communicate wirelessly and/or via the cloud.

In accordance with exemplary embodiments of the present invention, the device can be a defibrillator and/or monitor.

In accordance with yet another exemplary embodiment of the present invention, provided is a method that includes: monitoring environmental conditions and/or use of a battery; determining an amount of time remaining before the battery should be recharged or replaced, and/or an end of life condition of the battery; and displaying the amount of time remaining and/or the end of life condition on a display on the battery.

The foregoing form and other forms of the present invention as well as various features and advantages of the present invention will become further apparent from the following detailed description of various exemplary embodiments of the present invention read in conjunction with the accompanying drawings. The exemplary embodiments described herein and in the drawings are merely illustrative of the present invention rather than limiting, the scope of the present invention being defined by the appended claims and equivalents thereof.

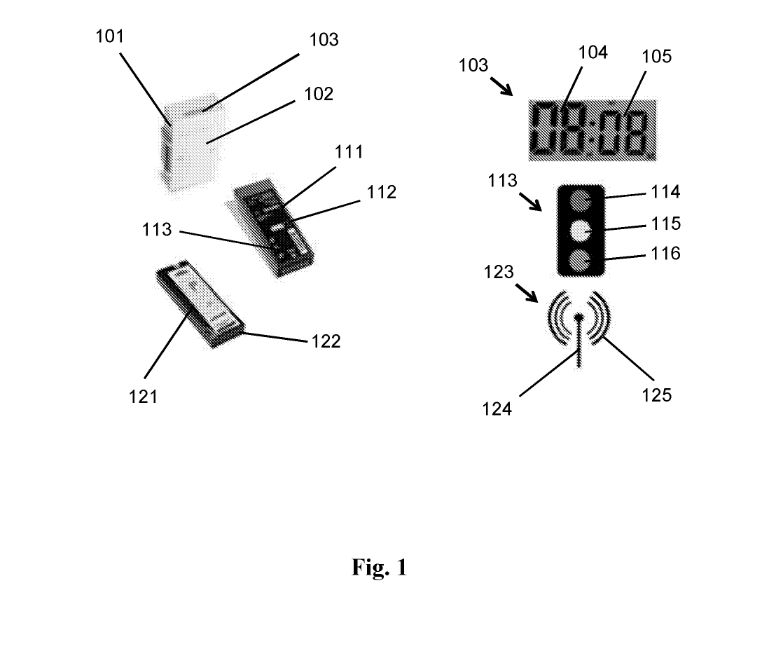

FIG. 1 illustrates exemplary embodiments of batteries in accordance with the present invention.

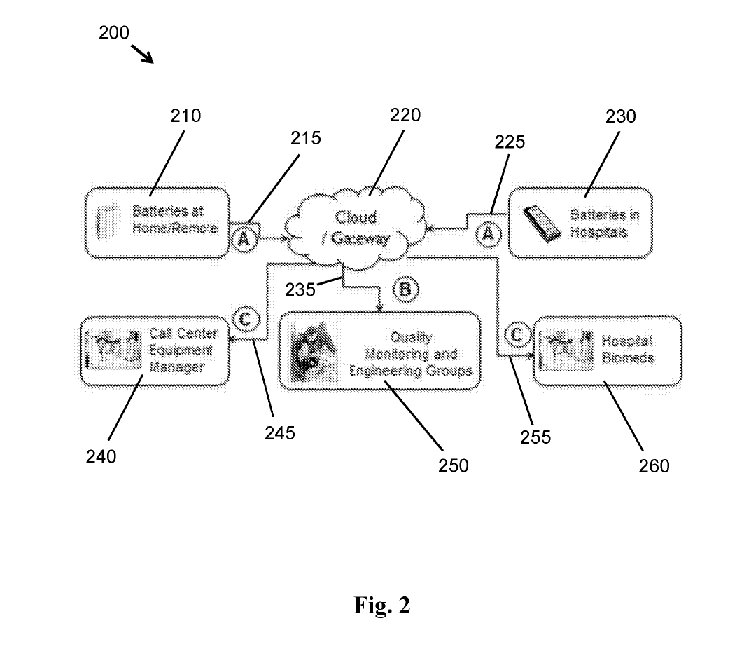

FIG. 2 illustrates an exemplary embodiment of a system in accordance with the present invention represented as a functional block diagram.

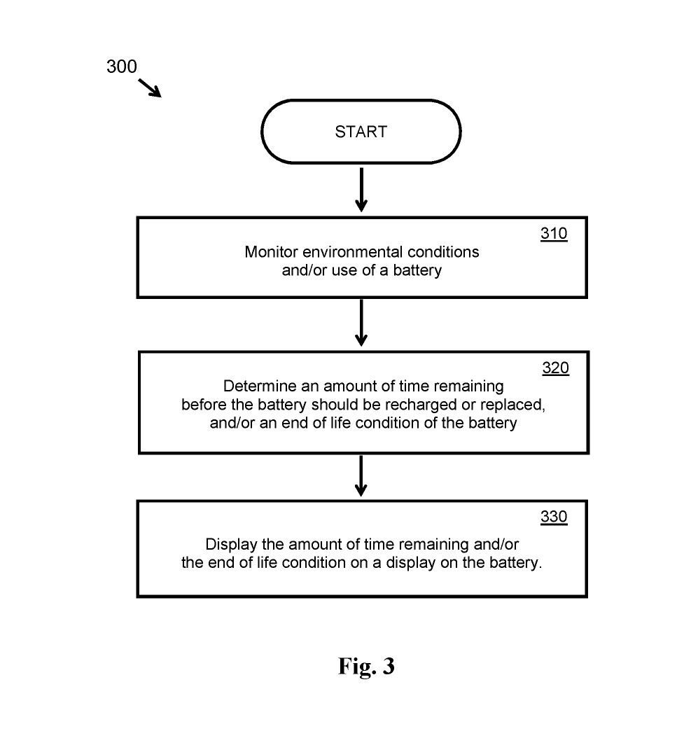

FIG. 3 illustrates a flow chart of an exemplary embodiment of a method in accordance with present invention.

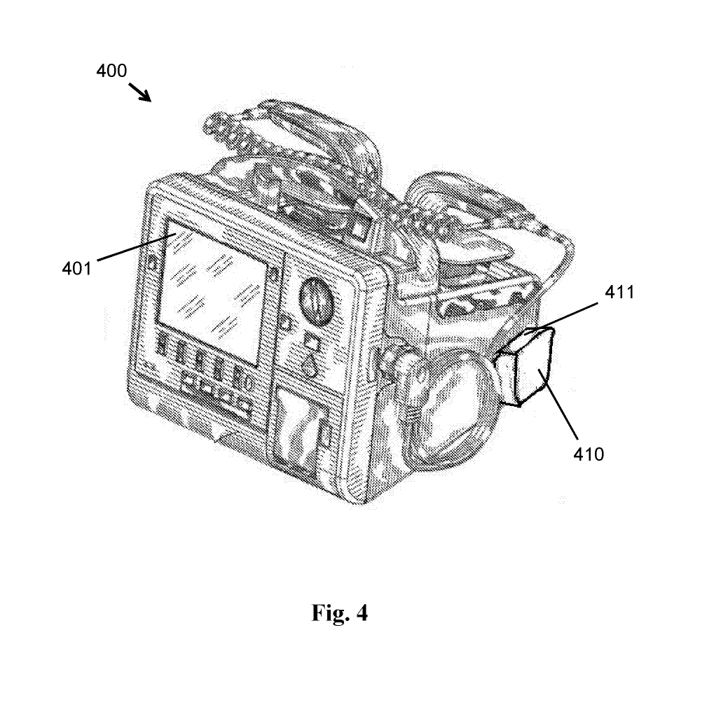

FIG. 4 is an illustration of an exemplary embodiment of a device in accordance with the present invention.

To further facilitate an understanding of the present invention, exemplary embodiments of the present invention are further described herein with reference to the accompanying figures.

Batteries may provide a LED “gas gauge” indicating percentage charged. For example, 5 LEDs may indicate percentage charge in 20% increments. While this may seem to be helpful, it may not be clear what the LEDs, or even the percentage of time means, especially when there are many different products and batteries being used, such as in a hospital or elsewhere. In a power outage or power interruption, this can add to the confusion, particularly when multiple battery powered products need to be managed.

In accordance with exemplary embodiments of the present invention, a display or read out on the battery indicating the exact (or estimated) amount of minutes the device can be used (e.g., with and/or without being connected to the device). While minutes of runtime may have been used/displayed on a device (e.g., computer) itself, it is believed that that minutes of runtime have never been displayed on a battery itself. Moreover, it is believed that exact (or estimated with a reasonable degree of accuracy) minutes of runtime have never before been used/displayed on a medical device, especially a defibrillator, which can have peak power demands in emergency situations for which accurate power management can be critical.

For example, according to certain exemplary embodiments of the present invention, the battery itself provides a universally clear indication of charge state e.g., “in minutes or hours” of product use. As shown in FIG. 1, for example, exemplary battery 101 has housing 102 which includes a display 103. As FIG. 1 further shows in the enlarged view of display 103 (on the right side of FIG. 1), the battery itself can have a runtime indicator in hours 104 and minutes 105 of the connected device.

In another exemplary embodiment of the present invention (which can be used in combination with, e.g., the first exemplary embodiment described herein), the battery itself indicates END OF LIFE condition so the user can know when the battery should be discarded/replaced based on, e.g., use and/or environmental conditions. This helps reduce costs (e.g., by not prematurely discarding a battery that still has life left in it). It also helps reduce the risk of battery failure (e.g., by not keeping a battery in service based on only time when the battery should have been discarded based on, e.g., use and/or environmental conditions and/or time).

Medical device manufacturers generally define a battery life in which the user must replace the battery based on only time (e.g., typically 2 to 3 years), regardless of use and environmental conditions. This typically requires tracking, monitoring, testing and generally becomes expensive. If one replaces the battery too soon, they are increasing their long term operational costs. If one replaces the battery too late, patient safety can be an issue. However, battery life is dependent on several factors, such as age, number of charge/discharge cycles, and environmental conditions such as temperature and humidity.

In accordance with certain exemplary embodiments of the present invention, technology and sensors are incorporated in the battery to measure, track and/or record such factors (e.g., environmental conditions, age and use model) to give an accurate (or reasonably accurate) end of life indication on the battery to the user. For example, if/when users abuse the battery and/or use the battery in harsh and/or demanding conditions, the battery would prompt the user to discard/replace with another battery (e.g., a new, reconditioned, or used battery that still has life left) more often/frequently, reducing/eliminating the risk of sudden battery failure. For users who are more conservative with batteries, they will generally have a longer time before being prompted to discard/replace the battery, thus reducing long term replacement costs/cost of ownership.

Further, in accordance with certain exemplary embodiments of the present invention, provided on the battery can be a tricolored LED and/or text display indicating the following, e.g.:
Green>80% life left
Yellow<20% life left. Replace soon
Red<10% life left. Replace Immediately
   These color changes can be based on age of battery, number of charge/discharge cycles, use temperature and/or other environmental inputs, for example.

With reference again to FIG. 1, for example, exemplary battery 111 has a housing 112 which includes a battery end-of-life indicator 113. As shown in the enlarged view of the battery end-of-life indicator 113 (on the right side of FIG. 1), the indicator 113 can have three colored lights (e.g., LEDs) that provide an indication of the battery's life. The top LED 114 can be illuminated in the color red, indicating that the battery should be replaced immediately. The middle LED can be illuminated in the color yellow, indicating that the battery's end of life is near. The bottom LED can be illuminated in the color green, indicating that the battery is in good condition.

In yet another exemplary embodiment of the present invention (which can be used in combination with, e.g., the first and/or second exemplary embodiments described herein), the battery itself has the ability to notify, e.g., call centers or the biomed department of a hospital of low battery conditions (including, e.g., indicating a device is unplugged or not charging a battery), battery fault, and/or battery end of life condition along with a GPS location.

In accordance with certain exemplary embodiments of the present invention, wireless communication and/or cloud technology is incorporated into the battery/device itself to notify/alert a remote user/manager to a low battery indication, battery faults, and end of life conditions (e.g., detected by the battery in accordance with the first and/or second exemplary embodiments described herein) along with its GPS location. For example, a typical hospital may have a few hundred different pieces of equipment and a few thousand different batteries distributive throughout the hospital, emergency vehicles (e.g., ambulances), home and/or other remote sites. However, as one having ordinary skill in the art shall appreciate in view of the teachings provided herein, this exemplary embodiment is certainly not limited to hospital environments. For example, this exemplary embodiment can be applied for any facility that may have multiple (medical) devices and/or batteries (e.g., doctor's office, medical labs, surgical centers, clinics, airports, airplanes, cruise ships, trains, office buildings, government buildings, public buildings, schools, etc.) The batteries can be programmed/configured to communicate/notify the indication, etc. to one or more predetermined locations (on- and/or off-site, physical and/or virtual) of the device/battery owner/operator and/or manufacturer/supplier, for example.

Referring again to FIG. 1, exemplary battery 121 has housing 122 which includes wireless communication and/or cloud technology 123. Wireless communication and/or cloud technology 123 can include a transmitter/transceiver/transponder 124 to transmit wireless signals 125. In accordance with exemplary embodiments of the present invention, the battery itself notifies call centers or biomed departments of hospitals of, e.g., low battery conditions, device being unplugged, battery not charging, battery fault, end of life condition, and GPS location.

Having the ability to monitor “battery health” can be a critical piece to monitoring, maintaining batteries, and being prepared when emergency backup power is needed. One example of how exemplary embodiments of the present invention can be extremely useful/beneficial (and (potentially) lifesaving) is in defibrillator application(s), e.g., Advanced Life Support, Basic Life Support, Automatic External Defibrillator (AED), pre-hospital, in-hospital, non-hospital, including public, private and military use/environments where battery power can be important or even critical for product operation. It can be critical that batteries work, known charged state and remaining life is accurate, and problems are detected prior to an emergency event during which the battery may be relied.

For example, defibrillators and other Advanced Life Support Devices are typically exposed to a wide range of end user use models and environmental conditions, which can range from a simple/relatively stable hospital environment (where the product is stationary and is in a well-controlled environment) to an EMS/Fire Truck/Battle field (where the product is highly mobile and often exposed to extreme environmental conditions).

As indicated above, because of the wide range of use models and product locations, it can be difficult to manage batteries and monitor large sample size and environmental conditions to develop accurate predictive models to prevent failures. In addition, because of the often critical nature of, e.g., defibrillators and other Advanced Life Support Devices, it can be imperative to collect this data non-invasively as to not interrupt critical care to the patient.

In accordance with exemplary embodiments of the present invention, data, including real-time or near real-time environmental data, can be collected remotely, stored and/or transmitted (e.g., wirelessly and/or via the cloud) to enable development of predictive failure models for an individual battery/product/device and/or over an entire population thereof, and to provide real-time alerts for battery issues so customized preventive maintenance feedback can be given to an end user, manufacturer, supplier, etc.

For example, FIG. 2 shows a system 200 in accordance with certain exemplary embodiments of the present invention. Battery system and environmental data are collected and sent via commination links 215 and 225 to the cloud 220 from local sites 230 (e.g., hospitals) and remote sites 210 (e.g., homes). The data can be sent from the cloud 220 via communication link 235 to quality monitoring and engineering groups 250 of, e.g., the device and/or battery manufacturer. Call centers 240 and hospital biomed departments 260 receive via communication links 245 and 255 notification of, e.g., low battery conditions, device unplugged, battery not charging, battery fault, end-of-life condition, and GPS location.

FIG. 3 illustrates a flow chart of an exemplary embodiment of a method 300 in accordance with present invention. As shown in FIG. 3, the exemplary method includes at step 310 monitoring environmental conditions and/or use of a battery. At step 320, the exemplary method includes determining an amount of an amount of time remaining before the battery should be recharged or replaced, and/or an end of life condition of the battery. Further, at step 330, the exemplary method includes displaying the amount of time remaining and/or the end of life condition on a display on the battery.

FIG. 4 is an illustration of an exemplary embodiment of a device in accordance with the present invention. As shown in FIG. 4, exemplary device 400 can be an external defibrillator. In this example and for illustrative purposes, device 400 is shown with battery 410 having display 411 coupled to a side of device 400. One having ordinary skill in the art shall appreciate, however, that batteries are often integrated within the housing of a device and accessible from the back or bottom of the device. Thus, in accordance with exemplary embodiments of the present invention, the battery information displayed on the battery display as disclosed and described herein can also be displayed on a main display 401 of the device.

While this invention has been described primarily with respect to medical devices and, in particular, defibrillators/monitors, such as in-hospital defibrillators/monitors (e.g., used by hospital personnel) and/or pre-hospital defibrillators/monitors (e.g., used by EMS personnel), one having ordinary skill in the art shall appreciate in view of the teachings provided herein that exemplary embodiments of the present invention can be implemented in other medical devices, including, but not limited to, patient monitors (e.g., ECG monitors), automatic external defibrillators (AEDs) and/or other defibrillators. Further, one having ordinary skill in the art shall appreciate in view of the teachings provided herein that exemplary embodiments of the present invention can be used with batteries/devices in non-medical (device) applications, including especially any application where battery maintenance and reliability can be particularly important, such as may be the case with certain battery powered (backed-up) communication, navigation and/or propulsion equipment, for example. Indeed, exemplary embodiments of the present invention implemented in batteries as may be used in/with these other types of products are specifically contemplated and considered to be within the scope of the present invention.

Further, as one having ordinary skill in the art will appreciate in view of the teachings provided herein, features, elements, components, etc. described in the present disclosure/specification and/or depicted in the appended Figures may be implemented in various combinations of hardware and software, and provide functions which may be combined in a single element or multiple elements. For example, the functions of the various features, elements, components, etc. shown/illustrated/depicted in the Figure can be provided through the use of dedicated hardware as well as hardware capable of executing software in association with appropriate software. When provided by a processor, the functions can be provided by a single dedicated processor, by a single shared processor, or by a plurality of individual processors, some of which can be shared and/or multiplexed. Moreover, explicit use of the term “processor” or “controller” should not be construed to refer exclusively to hardware capable of executing software, and can implicitly include, without limitation, digital signal processor (“DSP”) hardware, memory (e.g., read only memory (“ROM”) for storing software, random access memory (“RAM”), non-volatile storage, etc.) and virtually any means and/or machine (including hardware, software, firmware, combinations thereof, etc.) which is capable of (and/or configurable) to perform and/or control a process.

Moreover, all statements herein reciting principles, aspects, and exemplary embodiments of the present invention, as well as specific examples thereof, are intended to encompass both structural and functional equivalents thereof. Additionally, it is intended that such equivalents include both currently known equivalents as well as equivalents developed in the future (e.g., any elements developed that can perform the same or substantially similar functionality, regardless of structure). Thus, for example, it will be appreciated by one having ordinary skill in the art in view of the teachings provided herein that any block diagrams presented herein can represent conceptual views of illustrative system components and/or circuitry embodying the principles of the invention. Similarly, one having ordinary skill in the art should appreciate in view of the teachings provided herein that any flow charts, flow diagrams and the like can represent various processes which can be substantially represented in computer readable storage media and so executed by a computer, processor or other device with processing capabilities, whether or not such computer or processor is explicitly shown.

Furthermore, some exemplary embodiments of the present invention can take the form of a computer program product accessible from a computer-usable and/or computer-readable storage medium providing program code and/or instructions for use by or in connection with, e.g., a computer or any instruction execution system. In accordance with the present disclosure, a computer-usable or computer readable storage medium can be any apparatus that can, e.g., include, store, communicate, propagate or transport the program for use by or in connection with the instruction execution system, apparatus or device. Such exemplary medium can be, e.g., an electronic, magnetic, optical, electromagnetic, infrared or semiconductor system (or apparatus or device) or a propagation medium. Examples of a computer-readable medium include, e.g., a semiconductor or solid state memory, magnetic tape, a removable computer diskette, a random access memory (RAM), a read-only memory (ROM), flash (drive), a rigid magnetic disk and an optical disk. Current examples of optical disks include compact disk-read only memory (CD-ROM), compact disk-read/write (CD-R/W) and DVD. Further, it should be understood that any new computer-readable medium which may hereafter be developed should also be considered as computer-readable medium as may be used or referred to in accordance with exemplary embodiments of the present invention and disclosure.

Having described preferred and exemplary embodiments of automated battery indication and feedback systems based on environmental conditions and use data for improved battery/device management and reliability, (which embodiments are intended to be illustrative and not limiting), it is noted that modifications and variations can be made by persons having ordinary skill in the art in view of the teachings provided herein, including the appended Figures and claims. It is therefore to be understood that changes can be made in/to the preferred and exemplary embodiments of the present disclosure which are within the scope of the present invention and exemplary embodiments disclosed and described herein.

Moreover, it is contemplated that corresponding and/or related systems incorporating and/or implementing the device or such as may be used/implemented in a device in accordance with the present disclosure are also contemplated and considered to be within the scope of the present invention. Further, corresponding and/or related method for manufacturing and/or using a device and/or system in accordance with the present disclosure are also contemplated and considered to be within the scope of the present invention.

1. A battery, comprising:
a display indicating an amount of time the battery can be used before needing to be at least one of recharged or replaced.

2. The battery of claim 1, wherein the amount of time is displayed in minutes.

3. The battery of claim 1, wherein the display provides an end of life indicator including a tricolored LED.

4. The battery of claim 3, wherein the tricolored LED includes at least one of:
a green LED indicating the battery is in a good condition;
a yellow LED indicating the battery is nearing an end of life; or
a red LED indicating an immediate replacement of the battery.

5. The battery of claim 1 further comprising sensors for at least one of measuring or monitoring environmental conditions, wherein the battery determines the amount of time and an end of life indication based on at least one of the environmental conditions or usage.

6. The battery of claim 1 further comprising a global positioning system transponder.

7. The battery of claim 1 further comprising a communication link for transmitting data to at least one of a central location, a specified location or a pre-selected location.

8. The battery of claim 7, wherein the data includes the amount of time the battery can be used before needing to be at least one of recharged or replaced.

9. A system comprising:
a device; and
a battery, coupled to the device and configured to power the device,
wherein the battery comprises a display indicating an amount of time the battery can be used to power the device before needing to be at least one of recharged or replaced.

10. The system of claim 9, wherein the display is configured to display the amount of time in minutes.

11. The system of claim 9, further comprising an end of life indicator including a tricolored LED, wherein the tricolored LED includes at least one of:
a green LED indicating the battery is in a good condition;
a yellow LED indicating the battery is nearing an end of life; or
a red LED indicating an immediate replacement of the battery.

12. The system of claim 9, wherein the display is configured to display an end of life condition as a number of recharge cycles remaining before the battery should be replaced.

13. The system of claim 9, wherein the display is configured to display an end of life condition as an indication that the battery should be replaced.

14. The system of claim 9, wherein the battery determines the amount of time based on at least one of environmental conditions or use.

15. The system of claim 14, wherein the battery is structured and configured to at least one of measure or monitor use.

16. The system of claim 9 further comprising a communication link for transmitting data to at least one of a central location, a specified location or a pre-selected location.

17. The system of claim 16, wherein the communication link is coupled directly to the battery.

18. The system of claim 17, wherein the communication link is structured and configured to communicate at least one of wirelessly or via the cloud.

19. The system of claim 9, wherein the device is a defibrillator.

20. A method, comprising:
monitoring at least one of environmental conditions or use of a battery;
determining at least one of (i) an amount of time remaining before the battery should be recharged or replaced, or (ii) an end of life condition of the battery; and
displaying at least one of the amount of time remaining or the end of life condition on a display on the battery.
